(19) United States
(12) Patent Application Publication
(10) Pub. No.: US 2023/0190115 A1
(43) Pub. Date: Jun. 22, 2023

(54) PULSE OXIMETER

(71) Applicant: Micron Technology, Inc., Boise, ID (US)

(72) Inventor: Kathryn H. RUSSO, Boise, ID (US)

(73) Assignee: Micron Technology, Inc., Boise, ID (US)

(21) Appl. No.: 17/556,831

(22) Filed: Dec. 20, 2021

(51) Int. Cl. A61B 5/0205; A61B 5/00; A61B 5/1455

(52) U.S. Cl. CPC A61B 5/0205; A61B 5/6826; A61B 5/14552; A61B 2560/0233; A61B 5/02427

(57) ABSTRACT

A pulse oximetry system including a housing operable to interface with a digit of a user, a light emitter, and first and second light receivers. The light emitter is positioned adjacent to an inside surface of the housing and operable to emit a light, the light configured to transmit through tissue of the digit of the user, wherein some of the light is scattered by or reflected off the tissue of the digit of the user. The first light receiver is positioned adjacent to an inside surface of the housing opposite from the previously mentioned inside surface and operable to detect light that is transmitted through the tissue of the digit of the user. The second light receiver adjacent to an inside surface of the housing and operable to detect light that is scattered by or reflected off the tissue of the digit of the user.

### FIELD

The present disclosure relates generally to apparatuses, systems, and methods for measuring oxygen saturation of blood. More specifically, the disclosure relates to apparatuses, systems, and methods for determining oxygen saturation of blood in a person or other mammal in which pigmentation affects light transmittance.

### BACKGROUND

Oxygen saturation of blood is critical for oxygen delivery and function of humans and mammals. More specifically, hemoglobin red blood cells binds oxygen (O₂) to deliver the oxygen throughout the body. Normal or healthy levels of hemoglobin-bound oxygen in arterial blood (SaO₂) is typically around 97% or 98%. SaO₂ is calculated as a ratio of the oxygenated hemoglobin concentration (HbO₂) to the total hemoglobin concentration in the blood (HbO₂+Hb):

i.e., (SaO₂)=(HbO₂)/((HbO₂)+(Hb)).

Pulse oximetry (SpO₂), also referred to as photoplethysmography (PPG), measures oxygen saturation by transmitting light to the skin of a subject. For example, transmittance-type oximetry finds a ratio of light that is transmitted through tissue at specific wavelengths during systole (i.e., the phase of the heartbeat when the ventricles pump blood from the heart into the arteries) and diastole (i.e., the phase of the heartbeat when the ventricles relax to allow refilling of blood into the ventricles). SpO₂ is generally considered to be equivalent to SaO₂. However, it has been found that SpO₂ is not indicative or an accurate representation of SaO₂ in populations with darker skin pigmentation. For example, transmittance-type pulse oximetry often gives erroneous oxygen saturation values for patients with darker skin and low oxygenation due to light scattering and reflection. This is because calibration curves for pulse oximetry have generally been calibrated with respect to skin with lighter pigmentation. Because the emitted light is scattered by darker skin pigmentation before being able to transmit through the tissue and because the calibration curves for determining SpO₂ have been calibrated using data from those with lighter skin pigmentation, those with darker skin pigmentation receive calculated SpO₂ readings that are not indicative of the true SaO₂ levels.

Furthermore, it is not possible to determine the appropriate calibration curve for SpO₂ values based solely on the observed skin color. This is because melanin, the protein which is present in the skin of those with darker skin pigmentation, has been found to contribute significantly to the scatter of light at the surface of and within the skin. Melanin varies in size, has various distribution density between individuals, and has various distribution density even within an individual at different skin locations. SpO₂ can further be affected by skin type and thickness. Thus, observed skin color is not an accurate basis for determining the appropriate calibration curve for calculating SpO₂ that is indicative of SaO₂.

### SUMMARY

A pulse oximetry system and method are provided for detecting SpO₂ levels for users with varying skin pigmentation.

According to one example (“Example 1”), a pulse oximetry system is provided, the pulse oximetry system including a housing operable to interface with a digit of a user; a light emitter positioned adjacent to an inside surface of the housing and operable to emit a light having at least one wavelength, the light configured to transmit through tissue of the digit of the user, wherein some of the light is scattered by or reflected off the tissue of the digit of the user; a first light receiver positioned adjacent to an inside surface of the housing opposite from the inside surface adjacent to which the light emitter is positioned and operable to detect light that is transmitted through the tissue of the digit of the user; and a second light receiver adjacent to an inside surface of the housing and operable to detect light that is scattered by or reflected off the tissue of the digit of the user.

According to another example (“Example 2”), further to Example 1, the first light receiver is operable to generate transmitted-light data in response to the light that is received through the tissue of the digit of the user and the second light receiver is operable to generate scattered-light and/or reflected light data based on the light that is received based on the light that is scattered by or reflected off the tissue of the digit of the user.

According to another example (“Example 3”), further to Example 2, the scattered-light and/or reflected light data is provided to select a calibration curve based on the scattered-light and/or reflected light data.

According to another example (“Example 4”), further to Example 3, the calibration curve is selected from a plurality of calibration curves based on the scattered-light and/or reflected light data.

According to another example (“Example 5”), the pulse oximetry system of Example 4 further includes a processor operable to determine an R value ([AC660]/[DC660])/([AC940]/[DC940]) in response to the transmitted-light data, the R value indicating an SpO₂ level via the calibration curve.

According to another example (“Example 6”), the pulse oximetry system of Example 2 further includes a transmitter operable to send the transmitted-light data and the scattered-light and/or reflected light data.

According to another example (“Example 7”), further to Example 6, the transmitter is a wireless transceiver.

According to another example (“Example 8”), the pulse oximetry system of Example 2 further includes a processor operable to receive the transmitted-light data and calculate an R value ([AC660]/[DC660])/([AC940]/[DC940]) and operable to receive the scattered-light and/or reflected light data and determine a calibration curve based on the scattered-light and/or reflected light data, the processor operable to determine an SpO₂ level based on the calculated R value and the calibration curve.

According to another example (“Example 9”), the pulse oximetry system of Example 8 further includes a memory operable to store a database of a plurality of calibration curves each relating a different profile of skin pigmentation.

According to another example (“Example 10”), further to Example 1, the light emitter includes a red light emitter and an infrared light emitter.

According to another example (“Example 11”), further to Example 1, the light emitter is operable to emit light having a wavelength in a range of 350-450 nm.

According to another example (“Example 12”), the pulse oximetry system of Example 1 further includes a pulse monitor

According to one example (“Example 13”), a pulse oximetry system includes a housing including a first portion and a second portion, the first and second portions operable to at least partially surround at least a portion of a user's digit; a light emitter positioned adjacent to the first portion of the housing; a first light receiver positioned adjacent to the second portion of the housing; and a second light receiver positioned adjacent to the first portion of the housing.

According to another example (“Example 14”), further to Example 13, the first light receiver is operable to generate transmitted-light data in response to light that is transmitted through the tissue of the user and the second light receiver is operable to generate scattered-light and/or reflected light data in response to light that is scattered by or reflected off the tissue of the user.

According to another example (“Example 15”), further to Example 13, the first light receiver is positioned opposite the light emitter.

According to another example (“Example 16”), further to Example 13, the light emitter is operable to emit red light and infrared light.

According to one example (“Example 17”), a method of taking a reading of an SpO₂ level of a user includes emitting light from a light emitter wherein a portion of light is transmitted through tissue of the user and a portion of light is scattered and/or reflected by the tissue of the user; detecting at least some of the portion of light transmitted through the tissue of the user by a first light receiver; detecting at least some of the portion of light scattered and/or reflected by the tissue of the user by a second light receiver; selecting a calibration curve from a plurality of calibration curves based on the portion of light scattered and/or reflected by the tissue of the user detected by the second light receiver; calculating an R value based on the light transmitted through the tissue of the user detected by the first light receiver; and determining the SpO₂ level of the user based on the R value with respect to the calibration curve selected from the plurality of calibration curves.

According to another example (“Example 18”), further to Example 17, emitting light from the light emitter includes emitting light in the red and infrared spectra.

According to another example (“Example 19”), further to Example 17, the method includes detecting a pulse of the user over a predetermined period of time, wherein calculating the R value is an average over the predetermined period of time.

According to another example (“Example 20”), further to Example 17, the method includes blocking ambient light from being received by the second light receiver.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic of a pulse oximetry system for determining SpO₂ levels of a user, in accordance with an embodiment.

FIG. 2A is an illustration of an embodiment of the pulse oximetry system of FIG. 1, including a housing and various components positioned on the housing, in accordance with an embodiment.

FIG. 2B is an illustration of an embodiment of the pulse oximetry system of FIG. 2A showing one arrangement of light emitters and light receivers, in accordance with an embodiment.

FIG. 3 is an exemplary method of determining an SpO₂ level of a user, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

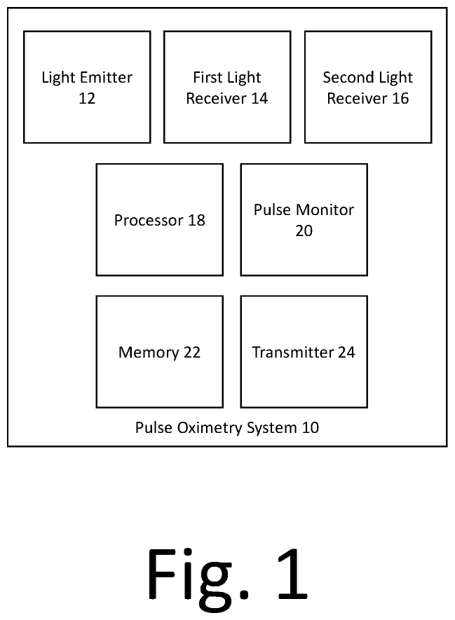

Referring to FIG. 1, a generalized schematic of a transmittance-type pulse oximetry system 10 is illustrated. The pulse oximetry system 10 measures the SpO₂ levels of a human or other mammals, the SpO₂ corresponding to the oxygen saturation of the arterial blood (i.e., SaO₂). The pulse oximetry system 10 is operable to obtain more accurate SpO₂ readings by normalizing readings from the pulse oximetry system based on light transmittance differences resulting from differences in skin pigmentation between users.

Pulse oximetry is implemented to measure light-absorption increase of the HbO₂ during systole. Specific wavelengths of the light are absorbed by the blood depending on the levels of oxygen saturation. For example, blood that is oxygen-rich (highly saturated) tends to absorb light having a wavelength of about 940 nm whereas blood that is oxygen-poor (low saturation) tends to absorb light having a wavelength of about 660 nm. Pulse oximetry typically compares the light-absorption of blood during systole and diastole. The ratio (R) used to determine blood saturation is calculated by finding the ratio between absorption of 660 nm light and 940 nm light. A baseline (DC component) is taken by measuring light absorption of tissue, venous blood, and non-pulsatile arterial blood (i.e., during diastole). This is used to determine the level of absorption of pulsatile arterial blood (AC component) when another measurement of transmittance is taken during systole (e.g., compares light absorption of tissue, venous blood, and pulsatile arterial blood to light absorption of tissue, venous blood, and non-pulsatile arterial blood). The absorption of pulsatile arterial blood at 660 nm is then compared to the light absorption of pulsatile arterial blood at 940 nm. Thus, R is determined by a ratio of ratios, shown below:

R=([AC₆₆₀]/[DC₆₆₀])/([AC₉₄₀]/[DC₉₄₀]) (Equation 1)

R is then used to provide a value for oxygen saturation as measured by pulse oximetry (SpO₂), which is generally representative of SaO₂. More specifically, a predefined calibration curve of SpO₂ levels is provided in relation to R values. Thus, an R value has a specific predetermined SpO₂ that corresponds to that R value. However, as previously noted, a single calibration curve does not represent the variability of transmittance through skins of varying pigmentations (e.g., does not account for scatter and reflection of light from skin with darker pigmentation).

The pulse oximetry system 10 is operable to provide a scatter value that is used to select an appropriate calibration curve for determining accurate SpO₂ values. The pulse oximetry system 10 interfaces with a user at a target tissue (e.g., a finger on a human) in order to take a reading of the user's SpO₂ levels. The pulse oximetry system 10 includes a light emitter 12 operable to emit light toward the target tissue, a first light receiver 14 (e.g., a photodetector) operable to detect light that has been transmitted through the target tissue, and a second light receiver 16 (e.g., photodetector) operable to detect light that has been scattered and/or reflected by the target tissue. The first light receiver 14 is operable to generate transmitted-light data in response to the light detected by the first light receiver 14. The second light receiver 16 is operable to generate scattered-light and/or reflected light data in response to the light detected by the second light receiver 16. The pulse oximetry system 10 further includes a processor 18 that is operable to receive the transmitted-light data and the scattered-light and/or reflected light data from the first and second light receivers 14, 16, respectively. The processor 18 is operable to calculate an R value based on the transmitted-light data as described above and known by those skilled in the art.

The pulse oximetry system 10 further includes a pulse monitor 20. The pulse monitor 20 is operable to determine the pulse of the user. As the pulse of the user is determined, the R value is able to be calculated as the SpO₂ levels can be determined based on the ratio of light transmittance during systole and diastole. In some embodiments, the processor 18 is operable to determine the pulse of the user via transmitted-light data via the first light receiver 14. The processor is operable to determine the pulse using the change in light transmittance through the tissue of the user over a period of time (e.g., spikes and dips in the calculated R values). In some embodiments, the pulse monitor 20 is a separate component from the first light receiver 14 and is operable to take the pulse of the user (e.g., electrical signals, pressure measurements, and so forth).

The pulse oximetry system further includes a memory 22. The memory 22 can be readable, writable, or both. The memory 22 includes a database including a plurality of calibration curves. The calibration curves can be generated and recorded separate from the use of the pulse oximetry system 10 by the user (e.g., prior to the user using the pulse oximetry system 10 for determining blood oxygen levels). Each calibration curve is associated with a light scattering and/or reflectance profile. For example, each calibration curve represents SpO₂ levels for various skin pigmentations. For example, the various calibration curves can be based on an average of various individuals with specific skin pigmentation. The skin pigmentation can be measured in a variety of methods, including but not limited to taking the light scattering and reflection data from the patients. The calibration curves are stored in the memory 22 including a specific profile for the each calibration curve that includes a scattering and reflection profile. The memory 22 may also be used for a variety of other purposes, including but not limited to storage of past SpO₂ reading for the user, user profiles, instructions, and so forth.

In use, the scattered-light and/or reflected light data is provided to determine a calibration curve based on the scattered-light and/or reflected light data. For example, the scattered light data that is generated by the second light receiver 16 is provided to the processor 18, which then accesses the database of calibration curves from the memory 22. The processor 18 selects a calibration curve from the database maintaining the plurality of predetermined calibration curves based on the scattered-light and/or reflected light data that matches or most closely matches one of the calibration curves. More specifically, the scattered-light and/or reflected light data can be matched with a similar or equivalent scattering and reflection profile of the calibration curve. When the scattered-light and/or reflected light data matches or closely matches a scattering and reflection profile of one of the calibration curves, that calibration curve is selected. The selected calibration curve is then used in connection with the transmitted-light data to determine the R value and SpO₂ levels, which is discussed hereafter.

In the event that the scattered-light and/or reflected light data does not match or substantially match a known calibration curve, the user is alerted that the no calibration curve was found. The pulse oximetry system 10 is operable to provide notification to a manufacturer that a calibration curve was not found matching the scattered-light and/or reflected light data. The manufacturer can then push updates with new or updated calibration curves to the pulse oximetry system.

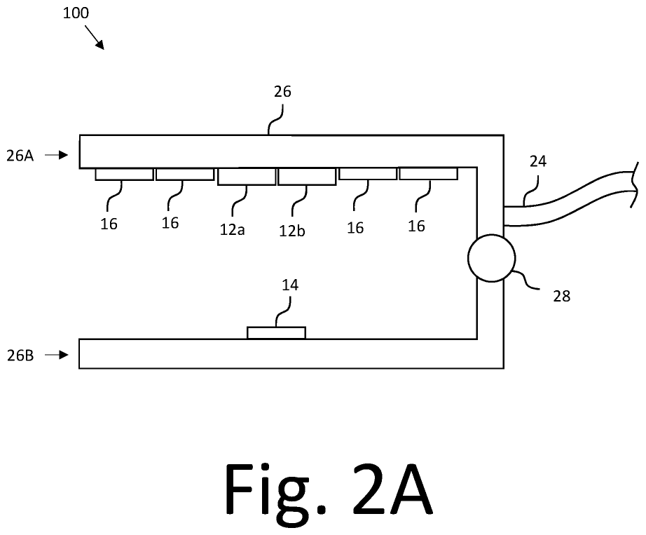

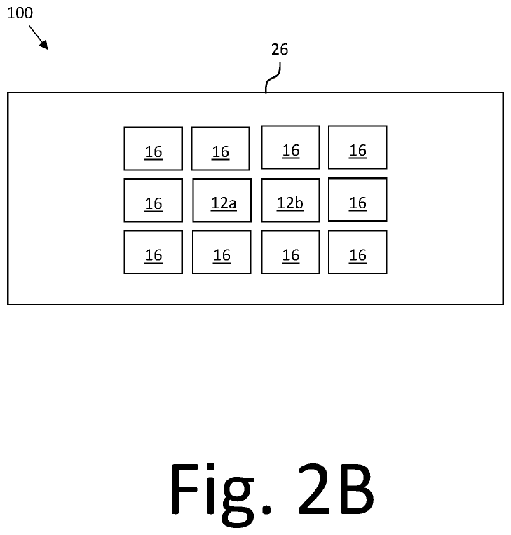

The pulse oximetry system 10 further includes a transmitter 24. The transmitter 24 is operable to transmit data to other components of the pulse oximetry system 10 (e.g., a monitor, computer, portable device, etc.) or to a device that is not part of the pulse oximetry system 10 with which the pulse oximetry system 10 interfaces (e.g., a cellular device via an application or an existing computer). The transmitter 24 include a wire for a wired connection or the transmitter may be a wireless transmitter such as a transceiver. Various wireless protocols may be implemented as known in the field, including but not limited to near field communication, radio frequency, WiFi®, Bluetooth®, and so forth. The data transmitted via the transmitter 24 may include a variety of information. The information transmitted may vary depending on the configuration of the pulse oximetry system 10. For example, in some embodiments, the pulse oximetry system 10 may include a physical interface member 100 (see FIG. 2A) that interfaces with the user and a computing member or a graphical user interface for computing and displaying the results of the reading. Any number of configurations may be implemented as is known in the field.

Referring to FIG. 2A, at least a portion of the pulse oximetry system 10 is illustrated. For example, FIG. 2A depicts the physical interface member 100 that includes a housing 26 that supports various components of the pulse oximetry system 10. The physical interface member 100 is formed to provide a physical interface with the user, for example the user's finger in order to take a reading of the user's SpO₂ levels. The housing 26 may include a first portion 26a and a second portion 26b that are coupled together at a hinge 28 that allows the housing to be inserted on and removed from the user's finger. In some embodiments, the housing 26 is operable to surround a portion of the user's finger so as to block out ambient light from entering into the interior space of the housing 26. Housings for pulse oximeters are well known in the industry and, therefore, no further discussion is needed.

In some embodiments, the light emitter 12 is positioned adjacent a first surface of the housing (e.g., the housing 26 supports the light emitter 12, the light emitter 12 is coupled to the housing 26, or the light emitter 12 is mounted on the housing 26). The light emitter 12 is operable to emit light as previously discussed. In some embodiments, the light emitter 12 is operable to emit light having at least one wavelength (e.g., a number of wavelengths or a range of wavelengths). In some embodiments, a plurality of light emitters 12 are implemented including a first light emitter 12a and a second light emitter 12b. The first and second light emitters 12a, 12b are operable to emit light having at least one wavelength (e.g., a number of wavelengths or a range of wavelengths). For example, the first light emitter 12a may emit light in a first range of wavelengths (e.g., 620 nm to 750 nm or red light) and the second light emitter 12b may emit light in a second range of wavelengths (e.g., 800 nm to 1 mm or infrared light). Other wavelengths and wavelength ranges are also contemplated including ultraviolet light which is absorbed by melanin and may distinguish between blood and dermis of a user. The wavelength ranges can include for example 350-450 nm. In some embodiments, the first and second light emitters 12a, 12b are spaced from each other on the housing 26. This allows light to be generated from different positions.

In some embodiments, the first light receiver 14 and the second light receiver 16 are positioned adjacent the housing (e.g., the housing 26 supports the first light receiver 14 and the second light receiver 16, the first light receiver 14 and the second light receiver 16 are coupled to the housing 26, or the first light receiver 14 and the second light receiver 16 are mounted on the housing 26). The first and second light receivers 14, 16 may be located at various positions about the housing 26. For example, the first light receiver 14 may be positioned substantially facing the light emitter 12. This positions the first light receiver 14 to receive light emitted by the light emitter that has transmitted through (e.g., traversed) the dermis of the user. For example, the light emitter 12 may be positioned on the first portion 26a of the housing 26 and the first light receiver 14 may be positioned on the second portion 26b of the housing 26 such that the first light receiver 14 is positioned opposing the light emitter 12. The first light receiver 14 is operable to detect (e.g., sense) the light that has been transmitted through the dermis and other tissue of the user. The first light receiver 14 may be operable to detect and differentiate various wavelengths or may be tuned to the specific wavelengths that are used to calculate O₂ saturation of the arterial blood. The first light receiver 14 provides data relating to the light received through the user's dermis to the processor 18 for calculating SpO₂. The processor 18 may be supported on the housing 26 or separate from the housing 26 (e.g., the processor of a computer, cellular device, or a secondary component of the pulse oximetry system 10). As previously described, the data may be transmitted via the transmitter 24.

In some embodiments, the housing 26 may support a plurality of first light receivers (not shown). In those embodiments implementing a plurality of first light receivers, the transmitted-light data that is generated by each of the first light receivers may be summed to determine total transmitted-light data. The total transmitted-light data may then be implemented in the SpO₂ of the user. Any number of first light receivers may be implemented in various embodiments. In other embodiments, the transmitted-light data that is generated by each of the first light receivers is averaged to determine an average transmitted-light data.

The second light receiver 16 is also supported on the housing 26. In some embodiments, the housing 26 supports a plurality of second light receivers 16. The second light receivers 16 may be positioned around the housing 26 so as to receive light that is scattered and/or reflected by the dermis or other tissues of the user. It is further understood that other pigmentation may also result in reflection and/or scattering of light, such as ink from tattoos, fingernail polish, and so forth. The second light receivers 16 may be positioned proximate the light emitter 12 to capture the reflected and/or scattered light. For example, the second light receivers 16 may be positioned on the same side of the housing 26 (e.g., first portion 26a). The second light receivers 16 are oriented in or positioned facing substantially the same direction of the light emitter 12 such that light that is emitted is reflected or scattered back from the user's tissue. The positioning and orientation of the second light receivers 16 relative to the light emitter 12 limits or prevents light from being received by the second light receivers 16 directly from the light emitter 12. In some embodiments, each of the second light receivers 16 is oriented toward a center of the housing 26, thus facilitating receipt of the reflected or scattered light that is reflected or scattered by the user's tissue that is positioned in the center of the housing 26 (e.g., in the interior space formed by the housing 26). In some embodiments, the second light receivers completely surround the light emitter 12, for example, as illustrated in FIG. 2B illustrating a planar view of the interior surface of a light emitter side of the housing 26.

In those embodiments implementing a plurality of second light receivers 16, the scattered-light and/or reflected light data that is generated by each of the second light receivers 16 may be summed to determine total scattered-light and/or reflected light data. The total scattered-light and/or reflected light data may then be used to determine a relevant calibration curve for the SpO₂ calculation. Any number of second light receivers 16 may be implemented in various embodiments. In other embodiments, the scattered-light and/or reflected light data that is generated by each of the second light receivers 16 is averaged to determine an average scattered-light and/or reflected light data.

In some embodiments, the housing 26 is operable to block ambient light from being received by the second light receiver 16. For example, the housing 26 can form a seal around the user's tissue such that ambient light is unable to enter into the space formed by the housing 26. Thus, when the light emitter 12 is not emitting light, there is little to no light being received by the light receivers 14, 16. In other embodiments, the light receivers 14, 16 take a baseline reading of the light being received prior to the light emitter 12 emitting light. The baseline reading represents the ambient light that is being sensed by the light receivers 14, 16 and is compared to the reading after the light emitter 12 emits light. This allows the pulse oximetry system 10 to obtain accurate reading for the first light receiver 14 and/or the second light receiver 16 for transmitted light and reflected or scattered light without having interference from any ambient light. It is understood that some embodiments may have a housing that blocks out ambient light as well as takes a baseline reading to filter out ambient light readings by the light receivers 14, 16 in the calculations.

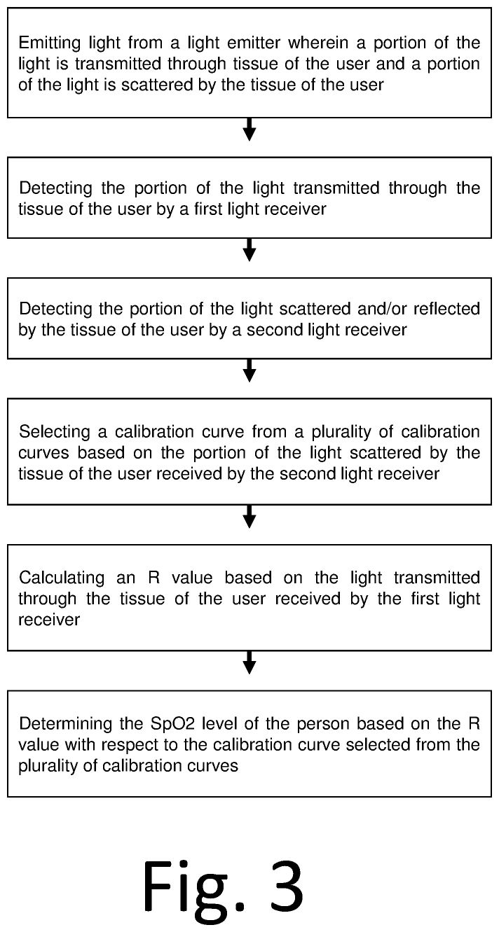

Referring now to FIG. 3, a method of obtaining an SpO₂ reading of a user is provided. The method includes emitting light from the light emitter 12 wherein a portion of the light is transmitted through tissue of the person and a portion of the light is scattered by the tissue of the user. As previously discussed, the light emitted by the light emitter 12 may include various properties including differing wavelengths that are each tuned to penetrate or be scattered and/or reflected by the tissue of the user. As various wavelengths are absorbed differently, in some embodiments, light may be emitted across a broader spectrum.

The method further includes detecting a portion of the light transmitted through the tissue of the user by the first light receiver 14 and detecting a portion of the light scattered and/or reflected by the tissue of the user by the second light receiver 16.

The method further includes selecting a calibration curve from a plurality of calibration curves based on the portion of the light scattered by the tissue of the person received by the second light receiver 16.

The method further includes calculating an R value based on the light transmitted through the tissue of the user received by the first light receiver 14 and determining the SpO₂ level of the user based on the R value with respect to the calibration curve selected from the plurality of calibration curves.

In some embodiments, the method includes emitting light from the light emitter includes emitting light in the red and infrared spectra.

In some embodiments, the method includes detecting a pulse of the person over a period of time, wherein calculating the R value is an average over the predetermined period of time.

In some embodiments, the method includes blocking ambient light from being received by the second light receiver.

It is understood that the method described herein may be implemented with the pulse oximetry system 10 discussed herein, or may be implemented with other embodiments contemplated herein. It is also understood that the methods may be performed with various independent tools that could be in combination considered a pulse oximetry system although some of the components are independent and do not form a single unit.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A pulse oximetry system, comprising:
a housing operable to interface with a digit of a user;
a light emitter positioned adjacent to an inside surface of the housing and operable to emit a light having at least one wavelength, the light configured to transmit through tissue of the digit of the user, wherein some of the light is scattered by or reflected off the tissue of the digit of the user;
a first light receiver positioned adjacent to an inside surface of the housing opposite from the inside surface adjacent to which the light emitter is positioned and operable to detect light that is transmitted through the tissue of the digit of the user; and
a second light receiver adjacent to an inside surface of the housing and operable to detect light that is scattered by or reflected off the tissue of the digit of the user.

2. The pulse oximetry system of claim 1, wherein the first light receiver is operable to generate transmitted-light data in response to the light that is received through the tissue of the digit of the user and the second light receiver is operable to generate scattered-light and/or reflected light data based on the light that is received based on the light that is scattered by or reflected off the tissue of the digit of the user.

3. The pulse oximetry system of claim 2, wherein the scattered-light and/or reflected light data is provided to select a calibration curve based on the scattered-light and/or reflected light data.

4. The pulse oximetry system of claim 3, wherein the calibration curve is selected from a plurality of calibration curves based on the scattered-light and/or reflected light data.

5. The pulse oximetry system of claim 4, further comprising a processor operable to determine an R value ([AC₆₆₀]/[DC₆₆₀])/([AC₉₄₀]/[DC₉₄₀]) in response to the transmitted-light data, the R value indicating an SpO₂ level via the calibration curve.

6. The pulse oximetry system of claim 2, further comprising a transmitter operable to send the transmitted-light data and the scattered-light and/or reflected light data.

7. The pulse oximetry system of claim 6, wherein the transmitter is a wireless transceiver.

8. The pulse oximetry system of claim 2, further comprising a processor operable to receive the transmitted-light data and calculate an R value ([AC₆₆₀]/[DC₆₆₀])/([AC₉₄₀]/[DC₉₄₀]) and operable to receive the scattered-light and/or reflected light data and determine a calibration curve based on the scattered-light and/or reflected light data, the processor operable to determine an SpO₂ level based on the calculated R value and the calibration curve.

9. The pulse oximetry system of claim 8, further comprising a memory operable to store a database of a plurality of calibration curves each relating a different profile of skin pigmentation.

10. The pulse oximetry system of claim 1, wherein the light emitter includes a red light emitter and an infrared light emitter.

11. The pulse oximetry system of claim 1, wherein the light emitter is operable to emit light having a wavelength in a range of 350-450 nm.

12. The pulse oximetry system of claim 1, further comprising a pulse monitor

13. A pulse oximetry system comprising:
a housing including a first portion and a second portion, the first and second portions operable to at least partially surround at least a portion of a user's digit;
a light emitter positioned adjacent to the first portion of the housing;
a first light receiver positioned adjacent to the second portion of the housing; and
a second light receiver positioned adjacent to the first portion of the housing.

14. The pulse oximetry system of claim 13, wherein the first light receiver is operable to generate transmitted-light data in response to light that is transmitted through the tissue of the user and the second light receiver is operable to generate scattered-light and/or reflected light data in response to light that is scattered by or reflected off the tissue of the user.

15. The pulse oximetry system of claim 13, wherein the first light receiver is positioned opposite the light emitter.

16. The pulse oximetry system of claim 13, wherein the light emitter is operable to emit red light and infrared light.

17. A method of taking a reading of an SpO₂ level of a user, the method comprising:
emitting light from a light emitter wherein a portion of light is transmitted through tissue of the user and a portion of light is scattered and/or reflected by the tissue of the user;
detecting at least some of the portion of light transmitted through the tissue of the user by a first light receiver;
detecting at least some of the portion of light scattered and/or reflected by the tissue of the user by a second light receiver;
selecting a calibration curve from a plurality of calibration curves based on the portion of light scattered and/or reflected by the tissue of the user detected by the second light receiver;
calculating an R value based on the light transmitted through the tissue of the user detected by the first light receiver; and
determining the SpO₂ level of the user based on the R value with respect to the calibration curve selected from the plurality of calibration curves.

18. The method of claim 17, wherein emitting light from the light emitter includes emitting light in the red and infrared spectra.

19. The method of claim 17, further comprising detecting a pulse of the user over a predetermined period of time, wherein calculating the R value is an average over the predetermined period of time.

20. The method of claim 17, further comprising blocking ambient light from being received by the second light receiver.
